SEQUENCE LISTING (8 sequences)

SEQ ID NO: 1 - protein, 238 aa, Artificial Sequence
  Dreiklang
VSKGEELFTGVVPILVELDGDVNGHKFSVSGEGEGDATYGKLTLKFICTTGKLPVPWPTLLTTIGYGLMCFARYPDHMKQHDFFKSAMPEGYVQERTIFFKDDGNYKTRAEVKFEGDTLVNRIELKGIDFKEDGNILGHKLEYNHDSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 2 - protein, 238 aa, Artificial Sequence
  PSFP2
VSKGEELFTGVVPILVELDGDVNGHKFSVSGEGEGDATYGKLTLKFVCTTGKLPVPWPTLLTTIGYGLMCFARYPDHMKQHDFFKSAMPEGYVQERTIFFKDDGNYKTRAEVKFEGDTLVNRIELKGIDFKEDGNILGHKLEYNHDSHNVYITADKQKNGIKVNFKIRHNIEDGSVQLADHYQQNTPIGDGPVLLPDNHYLSYQSALGKDPNEKRDHMVLLEFVTAAGITLGMDELYK

SEQ ID NO: 3 - protein, 238 aa, Artificial Sequence
  PSFP3
VSKGEELFTGVVPILVELDGDVNGHKFSVSGEGEGDATYGKLTLKFVCTTGKLPVPWPSLLTTIGYGLMCFARYPDHMKQHDFFKSAMPEGYVQERTIFFKDDGNYKTRAEVKFEGDTLVNRIELKGIDFKEDGNILGHKLEYNHDSHNVYITADKQKNGIKVNFKIRHNIEDGSVQLADHYQQNTPIGDGPVLLPDNHYLSYQSALGKDPNEKRDHMVLLEFVTAAGITLGTDELYK

SEQ ID NO: 4 - protein, 238 aa, Artificial Sequence
  PSFP4
VSKGEELFTGVVPILVELDGDVNGHKFSVSGEGEGDATYGKLTLKFVCTTGKLPVPWPSLLTTIGYGLQCFARYPDHMKQHDFFKSAMPEGYVQERTIFFKDDGNYKTRAEVKFEGDTLVNRIELKGIDFKEDGNILGHKLEYNHDSHNVYITADKQKNGIKVNFKIRHNIEDGSVQLADHYQQNTPIGDGPVLLPDNHYLSYQSALGKDPNEKRDHMVLLEFVTAAGITLGTDELYK

SEQ ID NO: 5 - DNA, 720 bp, Artificial Sequence
  PSFP2
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcgtctgcaccaccggcaagctgcccgtgccctggcccaccctcctgaccaccatcggatacggcctgatgtgcttcgcccgctatcccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaaccacgacagccacaacgtctatatcacggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgggcaaagaccccaacgagaagcgcgatcacatggtcctgctggagttcgtgaccgccgccgggatcaccctcggcatggacgagctgtacaagtaa

SEQ ID NO: 6 - DNA, 720 bp, Artificial Sequence
  PSFP3
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcgtctgcaccaccggcaagctgcccgtgccctggccctccctcctgaccaccatcggatacggcctgatgtgcttcgcccgctatcccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaaccacgacagccacaacgtctatatcacggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgggcaaagaccccaacgagaagcgcgatcacatggtcctgctggagttcgtgaccgccgccgggatcaccctcggcacggacgagctgtacaagtaa

SEQ ID NO: 7 - DNA, 720 bp, Artificial Sequence
  PSFP4
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcgtctgcaccaccggcaagctgcccgtgccctggccctccctcctgaccaccatcggatacggcctgcagtgcttcgcccgctatcccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaaccacgacagccacaacgtctatatcacggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgggcaaagaccccaacgagaagcgcgatcacatggtcctgctggagttcgtgaccgccgccgggatcaccctcggcacggacgagctgtacaagtaa

SEQ ID NO: 8 - protein, 17 aa, Artificial Sequence
  linker
GGSGGSGGSGGSGGQFQ

(19) United States
(12) Patent Application Publication
(10) Pub. No.: US 2018/0194816 A1
(43) Pub. Date: Jul. 12, 2018

(54) FLUORESCENT PROTEIN

(71) Applicant: OSAKA UNIVERSITY, Suita-shi, Osaka (JP)

(72) Inventors: Takeharu Nagai, Osaka (JP); Hiroki Takauchi, Osaka (JP); Yoshiyuki Arai, Osaka (JP); Masahiro Nakano, Osaka (JP)

(73) Assignee: OSAKA UNIVERSITY, Suita-shi, Osaka (JP)

(21) Appl. No.: 15/572,886

(22) PCT Filed: May 12, 2016
(86) PCT No.: PCT/JP2016/064132

§ 371 Date: Mar. 8, 2018

(30) Foreign Application Priority Data
May 12, 2015 (JP) 2015-097655

(51) Int. Cl. C07K 14/435; C12N 15/70; C12N 15/81; C12N 15/85; G01N 21/64; C12N 5/16; C12N 15/10

(52) U.S. Cl. CPC C07K 14/435; C12N 15/70; C12N 15/81; C07K 2319/60; G01N 21/6458; C12N 5/16; C12N 15/102; C12N 15/85

(57) ABSTRACT

A photo-switching fluorescent protein of a type in which wavelengths for switching fluorescence on and off and wavelength for fluorescence excitation are all independent of one another is provided, which has an amino acid sequence in which at least an S208G mutation is introduced into SEQ ID NO: 1. The fluorescent protein may have an amino acid sequence in which at least three mutations, I47V, M153T, and S208G, are introduced into SEQ ID NO: 1. The fluorescent protein may have an amino acid sequence in which at least five mutations, I47V, T59S, M153T, S208G, and M233T, are introduced into SEQ ID NO: 1. The fluorescent protein may have an amino acid sequence in which at least six mutations, I47V, T59S, M69Q, M153T, S208G, and M233T, are introduced into SEQ ID NO: 1.

### TECHNICAL FIELD

The present disclosure relates to a fluorescent protein and a DNA, vector, and transformant of said protein as well as an imaging method using the same.

### BACKGROUND ART

Fluorescent proteins have allowed live cell imaging to be performed easily and thereby various structures and functions in the cell have been made clear. In recent years, a super-resolution method using a fluorescent protein whose fluorescence can be reversibly photo-switched (a reversibly photo-switchable fluorescent protein, RSFP) has been developed, which has made it possible to perform imaging beyond the diffraction limit of an optical microscope. Examples of the RSFP include: 1) a negative photo-switching type that switches from a non-fluorescent state to a fluorescent state by irradiation with light having a specific wavelength that does not cause fluorescence excitation and switches from a fluorescent state to a non-fluorescent state by irradiation with light used for fluorescence excitation (for example, Dronpa (Patent Document 1), rsEGFP), 2) a positive photo-switching type that switches from a non-fluorescent state to a fluorescent state by irradiation with light used for fluorescence excitation and switches from a fluorescent state to a non-fluorescent state by irradiation with light having a specific wavelength that does not cause fluorescence excitation (for example, Padron (Non-Patent Document 1)), and 3) a type in which wavelengths for switching fluorescence on and off and for fluorescence excitation are all independent of one another (for example, Dreiklang (Non-Patent Document 2)).

### PRIOR ART DOCUMENTS

### Patent Documents

[Patent Document 1] WO2005/113772

### Non-Patent Documents

[Non-Patent Document 11] Tanja Brakemann et al, J. Biol. Chem. 2010, 285:14603-14609
[Non-Patent Document 2] Tanja Brakemann et al, Nat Biotechnol 29 (2011):942-947

### SUMMARY OF THE INVENTION

### Problem to be Solved by the Invention

With respect to the photo-switching fluorescent protein of type 3) mentioned above, no modified type has been reported since Dreiklang has been reported. Although Dreiklang has advantages such as being applicable to super-resolution microscopy referred to as photo activated localization microscopy (PALM) and also to super-resolution microscopy other than PALM, it has a slow photo-switching speed and thus a slow temporal resolution at super-resolution has been a problem.

In one or more embodiments, the present disclosure provides a variant of a photo-switching fluorescent protein of a type in which wavelengths for switching fluorescence on and off and for fluorescence excitation are all independent of one another

### Means for Solving Problem

In one or more embodiments, the present disclosure relates to a fluorescent protein having an amino acid sequence in which at least an S208G mutation is introduced into an amino acid sequence of SEQ ID NO: 1.

In further one or more embodiments, the present disclosure relates to a fluorescent protein having an amino acid sequence in which at least three mutations, I47V, M153T, and S208G, are introduced into the amino acid sequence of SEQ ID NO: 1.

In further one or more embodiments, the present disclosure relates to a fluorescent protein having an amino acid sequence in which at least five mutations, 147V, T59S, M153T, S208G, and M233T, are introduced into the amino acid sequence of SEQ ID NO: 1.

In further one or more embodiments, the present disclosure relates to a fluorescent protein having an amino acid sequence in which at least six mutations, I47V, T59S, M69Q, M153T, S208G, and M233T are introduced into the amino acid sequence of SEQ ID NO: 1.

In further one or more embodiments, the present disclosure relates to a fusion protein containing a fluorescent protein according to the present disclosure.

In further one or more embodiments, the present disclosure relates to an imaging method using a fluorescent protein or fusion protein according to the present disclosure.

### Effects of the Invention

In one or more embodiments, the present disclosure can provide a photo-switching fluorescent protein of a type in which wavelengths for switching fluorescence on and off and for fluorescence excitation are all independent of one another the fluorescent protein having an improved rate of recovery from a non-fluorescent state to a fluorescent state due to thermal equilibrium. In further one or more embodiments, the present disclosure can provide a photo-switching fluorescent protein of a type in which wavelengths for switching fluorescence on and off and for fluorescence excitation are all independent of one another, the fluorescent protein having a rate of recovery from a non-fluorescent state to a fluorescent state due to thermal equilibrium that has been improved compared to that of Dreiklang (SEQ ID NO: 1).

In one or more embodiments, a fluorescent protein according to the present disclosure can improve the spatial resolution and the temporal resolution in super-resolution imaging.

### BRIEF DESCRIPTION OF DRAWINGS

FIG. 1 is an alignment of the amino acid sequences of Dreiklang (SEQ ID NO: 1), PSFP2 (SEQ ID NO: 2), PSFP3 (SEQ ID NO: 3), and PSFP4 (SEQ ID NO: 4).

FIG. 2 is a diagram showing five sites mutated from Dreiklang in PSFP3.

FIG. 3 is an example of graphs obtained by measuring the rate of recovery of fluorescence to the on state due to thermal equilibrium after light was irradiated to switch fluorescence off, with respect to PSFP2, PSFP3, PSFP4, and Dreiklang.

FIG. 4 is an example of graphs obtained by measuring the speed of photo-switching fluorescence from the on state to the off state, with respect to PSFP2, PSFP3, and Dreiklang

FIG. 5 is an example of graphs obtained by measuring changes with time of fluorescence by bleaching, with respect to PSFP 2 PSFP 3, PSFP 4, and Dreiklang.

FIG. 6 shows graphs illustrating examples of continuous photo-switching with respect to PSFP3 and Dreiklang.

FIG. 7 shows images illustrating examples obtained by observing the localization of fusion proteins of PSFP3.

FIG. 8 is a diagram for explaining the measurement principle of decoupled stochastic switching microscopy (DSSM).

FIG. 9 shows examples of the images obtained by full-field microscopy (left) and examples of the images obtained by super-resolution microscopy (DSSM) (right) of vimentin using PSFP3.

FIG. 10 shows images illustrating examples obtained by observing fluorescence photo-switching of a localized fusion protein of PSFP3.

FIG. 11 shows images illustrating other examples obtained by observing the localization of fusion proteins of PSFP3.

FIG. 12 is a diagram showing an outline of an example of observation schemes for time-lapse super-resolution imaging.

FIG. 13 shows images illustrating examples obtained by observing the time-lapse super-resolution imaging.

### DESCRIPTION OF PREFERRED EMBODIMENTS

The present disclosure is based on the findings that by introducing an S208G mutation into Dreiklang (SEQ ID NO 1) which is a photo-switching fluorescent protein of a type in which a wavelength for switching a non-fluorescent state to a fluorescent state, a wavelength for switching a fluorescent state to a non-fluorescent state, and a wavelength for fluorescence excitation all differ from one another, the rate of recovery from a non-fluorescent state to a fluorescent state (the speed at which fluorescence is spontaneously switched on from of) due to thermal equilibrium is three times higher than that of Dreiklang, and the speed at which a fluorescent state is switched to a non-fluorescent state by light irradiation (photo-switching speed from on to off) is two times higher than that of Dreiklang.

The present disclosure is also based on the findings that in a fluorescent protein (PSFP2) in which a total of three mutations including I47V and M153T in addition to an S208G mutation are introduced into Dreiklang, the rate of recovery from a non-fluorescent state to a fluorescent state due to thermal equilibrium is three times higher than that of Dreiklang, the speed at which a fluorescent state is switched to a non-fluorescent state by light irradiation is two times higher than that f Dreiklang, and furthermore, the intensity in a fluorescent state increases compared to the case where a single mutation, S208G, is introduced.

Furthermore, the present disclosure is also based on the finding that in a fluorescent protein in which two mutations, T598 and M233T, are introduced into the above-mentioned PSFP2 (i.e., a fluorescent protein in which five mutations, S208G, I47V, M153T, T59S, and M233T, are introduced into Dreiklang) (PSFP3), the speed at which a fluorescent state is switched to a non-fluorescent state by light irradiation is improved 3.7 times compared to that of Dreiklang.

The five mutation sites in PSFP3 are located at the sites in Dreiklang shown in FIG. 2. These mutation sites each are located apart from the β-barrel structure in which the Dreiklang chromophore is located, and it can be said that it is an unpredictable effect from technical common sense that the mutations located at these sites improve the rate of recovery from a non-fluorescent state to a fluorescent state due to thermal equilibrium and the speed at which a fluorescent state is switched to a non-fluorescent state by light irradiation. Moreover, the scope of the present invention and the effects of the present invention are not interpreted as being limited to the numerical values described as “x times” with respect to the effects described above or later.

Furthermore, the present disclosure is also based on the finding that in a fluorescent protein in which an M69Q mutation is introduced into the above-mentioned PSFP3 (i.e., a fluorescent protein in which six mutations, S208G, I47V, M153T, T59S, M233T, and M69Q, are introduced into Dreiklang) (PSFP4), the fluorescence photostability (suppression of fluorescence decay by bleaching) improves 2.8 times compared to that of Dreiklang.

### [Fluorescent Protein]

The fluorescent protein according to the present disclosure is a photo-switching fluorescent protein of a type in which a wavelength for switching a non-fluorescent state to a fluorescent state, a wavelength for switching a fluorescent state to a non-fluorescent state, and a wavelength for fluorescence excitation all differ from one another. Dreiklang, a protein comprising the amino acid sequence of SEQ ID NO: 1, is a photo-switching fluorescent protein of this type.

In the present disclosure, the term “photo-switching fluorescent protein” denotes, in one or more embodiments, a protein in which on and off of the fluorescent state (i.e., a fluorescent state and a non-fluorescent state) can be controlled by irradiation of two lights that are different in wavelength, and switching on and off can be repeatedly carried out

In the present disclosure, mutations represented by “X₁nX₂”, which is a general notation method for mutations, represent those in which the n-th amino acid residue X₁ (an amino acid residue in one letter code) in an amino acid sequence is substituted with an amino acid residue X₂ (an amino acid residue in one letter code).

In an aspect, the present disclosure relates to a fluorescent protein having an amino acid sequence in which at least an S208G mutation is introduced into the amino acid sequence of SEQ ID NO: 1.

Introduction of the S208G mutation improves the rate of recovery from a non-fluorescent state to a fluorescent state due to thermal equilibrium as compared to that obtained before the introduction of said mutation. In further one or more embodiments, introduction of the above-mentioned S208G mutation improves the rate of recovery from a non-fluorescent state to a fluorescent state due to thermal equilibrium and can improve the speed at which a fluorescent state is switched to a non-fluorescent state by light irradiation, as compared to those obtained before the introduction of said mutation.

Examples of one or more embodiments of the fluorescent protein according to the present disclosure include a fluorescent protein having an amino acid sequence in which at least an S208G mutation and at least one or two to five mutations selected from the group consisting of I47, T598, M69Q, M153T, and M233T are introduced into the amino acid sequence of SEQ ID NO: 1.

The fluorescent protein of the present embodiment can be a photo-switching fluorescent protein having a rate of recovery from a non-fluorescent state to a fluorescent state due to thermal equilibrium that is improved compared to that obtained before the introduction of said mutation.

Among the above-mentioned mutations, the mutation, I47V or M153T, or the mutations, I4V and M153T, may contribute to improving the fluorescence intensity. Among the above-mentioned mutations, the mutation, T598 or M233T, or the mutations, T598 and M233T, may contribute to improving the speed at which a fluorescent state is switched to a non-fluorescent state by light irradiation. Among the above-mentioned mutations, the M69Q mutation may contribute to improving the fluorescence photostability.

Examples of one or more embodiments of the fluorescent protein according to the present disclosure include a fluorescent protein having an amino acid sequence in which at least three mutations, I471, M153T, and S208G, are introduced into the amino acid sequence of SEQ ID NO: 1. The amino acid sequence in which the above-mentioned three mutations are introduced into SEQ ID NO: 1 is SEQ ID NO: 2 (PSFP2).

In the fluorescent protein of the present embodiment, introduction of the mutations, I47V and M153T in addition to the S208G mutation may improve the intensity in a fluorescent state as compared to the case where the 208G mutation alone has been introduced.

Examples of one or more embodiments of the fluorescent protein according to the present disclosure include a fluorescent protein having an amino acid sequence in which at least five mutations, I47V, T59S, M153T, S208G, and M233T, are introduced into the amino acid sequence of SEQ ID NO: 1. The amino acid sequence in which the above-mentioned five mutations are introduced into SEQ ID NO: 1 is SEQ ID NO: 3 (PSFP3).

In the fluorescent protein of the present embodiment, the mutations, T59S and M233T, are introduced in addition to the mutations, I47V, M153T, and S208G, and thereby the speed at which a fluorescent state is switched to a non-fluorescent state by light irradiation may be further improved as compared to that obtained before the introduction of the mutations, T59S and M233T.

There is a fluorescent protein having an amino acid sequence in which at least six mutations, I47V, T59S, M69Q, M153T, S208G, and M233T, are introduced into the amino acid sequence of SEQ ID NO: 1. The amino acid sequence in which the above-mentioned six mutations are introduced into SEQ ID NO: 1 is SEQ ID NO: 4 (PSFP4).

In the fluorescent protein of the present embodiment, the M69Q mutation is introduced in addition to the five mutations, I47V, T59S, M153T, S208G, and M233T, and thereby the fluorescence photostability may be improved as compared to that obtained before the introduction of the M69Q mutation.

In the present disclosure, the “rate of recovery from a non-fluorescent state to a fluorescent state due to thermal equilibrium”, the “speed at which a fluorescent state is switched to a non-fluorescent state by light irradiation”, and the “fluorescence photostability” can be measured and evaluated by the methods described in Examples.

In one or more embodiments, it is preferable that the fluorescent protein according to the present disclosure have a higher rate of recovery from a non-fluorescent state to a fluorescent state due to thermal equilibrium than that of Dreiklang (SEQ ID NO: 1). In one or more embodiments, it is preferable that the fluorescent protein according to the present disclosure have a higher speed at which a fluorescent state is switched to a non-fluorescent state by light irradiation than that of Dreiklang (SEQ ID NO: 1). In one or more embodiments, it is preferable that the fluorescent protein according to the present disclosure have a higher fluorescence photostability than that of Dreiklang (SEQ ID NO: 1).

In one or more embodiments, the fluorescent protein according to the present disclosure may have mutations other than the above-mentioned six mutations (I47V, T59S, M69Q, M153T, S208G, and M233T) within the range where the functions of a photo-switching fluorescent protein of a type in which a wavelength for switching a non-fluorescent state to a fluorescent state, a wavelength for switching a fluorescent state to a non-fluorescent state, and a wavelength for fluorescence excitation all differ from one another can be maintained. Examples of the mutations other than the above-mentioned six mutations include deletions, substitutions, and/or additions of one to several amino acids, where the expression “one to several” includes one to four, one to three, one to two, or one in one or more embodiments.

In one or more embodiments, even when the fluorescent protein according to the present disclosure includes mutations other than the above-mentioned six mutations, it is preferable that the fluorescent protein have a higher rate of recovery from a non-fluorescent state to a fluorescent state due to thermal equilibrium than that of Dreiklang (SEQ ID NO: 1).

In one or more embodiments, even when the fluorescent protein according to the present disclosure includes mutations other than the above-mentioned six mutations, it is preferable that the fluorescent protein have a higher speed at which a fluorescent state is switched to a non-fluorescent state by light irradiation than that of Dreiklang (SEQ ID NO: 1).

In one or more embodiments, even when the fluorescent protein according to the present disclosure includes mutations other than the above-mentioned six mutations, it is preferable that the fluorescent protein have a higher fluorescence photostability than that of Dreiklang (SEQ ID NO: 1).

In one or more embodiments, the fluorescent protein according to the present disclosure may be a protein synthesized by chemical synthesis or a recombinant protein produced by gene recombination technology. In one or more embodiments, examples of the method of producing a recombinant protein by the gene recombination technology include a method of producing a recombinant protein using a host transformed with an expression vector containing a gene that encodes a fluorescent protein according to the present disclosure.

In one or more embodiments, the fluorescent protein according to the present disclosure is a fusion protein in which the above-mentioned fluorescent protein according to the present disclosure is fused with another protein or peptide, a part of the fusion protein, which is said fluorescent protein, being capable of functioning as a photo-switching fluorescent protein in which a wavelength for switching a non-fluorescent state to a fluorescent state, a wavelength for switching a fluorescent state to a non-fluorescent state, and a wavelength for fluorescence excitation all differ from one another.

In one or more embodiments, in the fusion protein according to the present disclosure, it is preferable that the part thereof, which is said fluorescent protein, have a higher rate of recovery from a non-fluorescent state to a fluorescent state due to thermal equilibrium than that of Dreiklang (SEQ ID NO: 1).

In one or more embodiments, in the fusion protein according to the present disclosure, it is preferable that the part thereof which is said fluorescent protein, have a higher speed at which a fluorescent state is switched to a non-fluorescent state by light irradiation than that of Dreiklang (SEQ ID NO: 1).

In one or more embodiments, in the fusion protein according to the present disclosure, it is preferable that the part thereof, which is said fluorescent protein, have a higher fluorescence photostability than that of Dreiklang (SEQ ID NO: 1).

In the fusion protein according to the present disclosure, examples of the protein that is bound (fused) to the fluorescent protein according to the present disclosure include a signal sequence, an expression tag, or a protein (a linker sequence as required) in one or more non-limiting embodiments. In one or more embodiments, from the viewpoint of imaging, examples of the signal sequence and the protein include those that can be localized in cytoskeletons (microfilaments, intermediate filaments, and microtubules) and cell organelles (nuclei, endoplasmic reticula, Golgi bodies, mitochondria, endosomes, lysosomes, etc.) within cells.

Since the fluorescent protein according to the present disclosure has a high photo-switching speed and allows fluorescence to be read repeatedly, it can be used for super-resolution imaging ultra-high density optical memory, ultra-sensitive fluorescence imaging, etc. in one or more embodiments. Furthermore, in one or more embodiments, the fluorescent protein according to the present disclosure can be used as a fluorescent functional indicator a photoprotein, or a luminescent functional indicator using a photo-switching fluorescent protein.

### [DNA]

In an aspect, the present disclosure relates to a DNA that encodes a fluorescent protein according to the present disclosure or a fusion protein according to the present disclosure. In one or more embodiments, a recombinant vector containing the DNA of the present disclosure is introduced into a host and thereby the DNA of the present disclosure can express and produce a fluorescent protein according to the present disclosure. In one or more embodiments, the DNA of the present disclosure can be produced by, for example, PCR using a specific primer or a phosphoamidite method.

### [Vector]

In an aspect, the present disclosure relates to a vector capable of expressing a fluorescent protein according to the present disclosure or a fusion protein according to the present disclosure. In one or more embodiments, the vector of the present disclosure is a vector having a DNA of the present disclosure. In one or more embodiments, the vector of the present disclosure can be obtained by inserting the DNA of the present disclosure into a suitable vector. In one or more embodiments, the vector into which the DNA of the present disclosure is inserted is not particularly limited as long as it can replicate in a host, and examples thereof include a plasmid and a phage.

In one or more embodiments, examples of the plasmid include plasmids derived from Escherichia coli, plasmids derived from Bacillus subtilis, and plasmids derived from yeasts.

### [Transformant]

In an aspect, the present disclosure relates to a transformant that expresses a fluorescent protein according to the present disclosure or a fusion protein according to the present disclosure. In one or more embodiments, the transformant of the present disclosure is a cell that expresses a fluorescent protein according to the present disclosure or a fusion protein according to the present disclosure, or a tissue, organ, or living body including said cell. Furthermore, in one or more embodiments, the present disclosure relates to a transformant having a DNA or recombinant vector of the present disclosure. In one or more embodiments, the transformant of the present disclosure can be prepared by introducing the DNA or recombinant vector of the present disclosure into a host.

In one or more embodiments, examples of the host include commonly used known microorganisms and cultured cells. In one or more embodiments, examples of the microorganisms include Escherichia coli and yeasts. In one or more embodiments, examples of the cultured cells include animal cells (for example, CHO cells, HEK-293 cells, or COS cells) and insect cells (for example, BmN4 cells).

### [Imaging Method]

In an aspect, the present disclosure relates to an imaging method using a fluorescent protein according to the present disclosure or a fusion protein according to the present disclosure, a DNA of the present disclosure, or a vector of the present disclosure. In one or more embodiments, the imaging method of the present disclosure includes introducing a fluorescent protein according to the present disclosure or a fusion protein according to the present disclosure into, for example, a cell, photo-switching the fluorescent protein according to the present disclosure or the fusion protein according to the present disclosure to switch fluorescence on and/or off, and/or detecting the fluorescence signal of the fluorescent protein according to the present disclosure or the fusion protein according to the present disclosure. In one or more embodiments, the imaging method of the present disclosure is super-resolution imaging, and examples thereof include, in one or more embodiments, decoupled stochastic switching microscopy (DSSM), photoactivated localization microscopy (PALM), stochastic optical reconstruction microscopy (STORM), reversible saturable optical fluorescence transition (RESOLFT), and super-resolution optical fluctuation imaging (SOFI). Since the fluorescent protein according to the present disclosure has an improved speed at which fluorescence is spontaneously switched on (the rate of recovery from a non-fluorescent state to a fluorescent state due to thermal equilibrium), it can be preferably used for DSSM, which is a simple super-resolution imaging method, in one or more embodiments.

### [Photochromic Material]

Since the fluorescent protein according to the present disclosure exhibits a photochromism effect, in one or more embodiments, it can be used as a photochromic material that can be applied to optical recording media such as CDs, DVDs, holographic recording media, and smart cards, display elements such as billboards, fluorescent screens, TVs, and computer monitors, or for lenses, biosensors, biochips, or photochromic fiber materials.

The present disclosure further relates to one or more non-limiting embodiments described below

[1] A fluorescent protein having an amino acid sequence in which at least an S208G mutation is introduced into an amino acid sequence of SEQ ID NO: 1.
[2] The fluorescent protein according to item 1, wherein the fluorescent protein has an amino acid sequence in which at least three mutations, I47n M153T, and S208G, are introduced into the amino acid sequence of SEQ ID NO: 1.
[3] The fluorescent protein according to item 1 or 2, wherein the fluorescent protein has an amino acid sequence in which at least five mutations, I47V, T59S, M153T, S208G, and M233T, are introduced into the amino acid sequence of SEQ ID NO: 1.
[4] The fluorescent protein according to any one of items 1 to 3, wherein the fluorescent protein has an amino acid sequence in which at least six mutations, I47V, T59S, M69Q M153T, S208G, and M233T, are introduced into the amino acid sequence of SEQ ID NO: 1.
[5] The fluorescent protein according to any one of items 1 to 4, wherein the fluorescent protein has an amino acid sequence of any one of SEQ ID NOS: 2 to 4.
[6] A fluorescent protein, the fluorescent protein having an amino acid sequence in which one to several amino acids are deleted, substituted, and/or added in the amino acid sequence of a protein according to any one of items 1 to 5, and in the fluorescent protein, a wavelength for switching a non-fluorescent state to a fluorescent state, a wavelength for switching a fluorescent state to a non-fluorescent state, and a wavelength far fluorescence excitation all differing from one another and the rate of recovery from a non-fluorescent state to a fluorescent state due to thermal equilibrium being higher than that of a protein comprising an amino acid sequence of SEQ ID NO: 1.
[7] A fusion protein in which a fluorescent protein according to any one of items 1 to 6 is fused, in a part of the fusion protein, which is said fluorescent protein, a wavelength for switching a non-fluorescent state to a fluorescent state, a wavelength for switching a fluorescent state to a non-fluorescent state, and a wavelength for fluorescence excitation all differing from one another and the rate of recovery from a non-fluorescent state to a fluorescent state due to thermal equilibrium being higher than that of a protein comprising an amino acid sequence of SEQ ID NO: 1.
[8] A DNA comprising a base sequence that encodes a protein according to any one of items 1 to 7.
[9] A vector being capable of expressing a protein according to any one of items 1 to 7 or comprising a DNA according to item 8.
[10] A transformant that expresses a protein according to any one of items 1 to 7.
[11] An imaging method using a protein according to any one of items 1 to 7, a DNA according to item 8, a vector according to item 9, or a transformant according to item 10.
[12] The imaging method according to item 11, wherein the imaging method is super-resolution fluorescence microscopy imaging.
[13] A photochromic material comprising a protein according to any one of items 1 to 7.

Hereinafter the present disclosure will be described in further details using examples but they are illustrative and the present disclosure is not limited to these examples.

### EXAMPLES

### [Production of Photo-Switching Fluorescent Proteins (PSFP)]

Three mutations, Mutations 1, 3, and 4 described below, were introduced into Dreiklang (a protein consisting of an amino acid sequence of SEQ ID NO: 1) which is a photo-switching fluorescent protein in which a wavelength far switching a non-fluorescent state to a fluorescent state, a wavelength for switching a fluorescent state to a non-fluorescent state, and a wavelength for fluorescence excitation all differ from one another. Thus, a photo-switching fluorescent protein PSFP2 was produced (a protein consisting of an amino acid sequence of SEQ ID NO: 2).

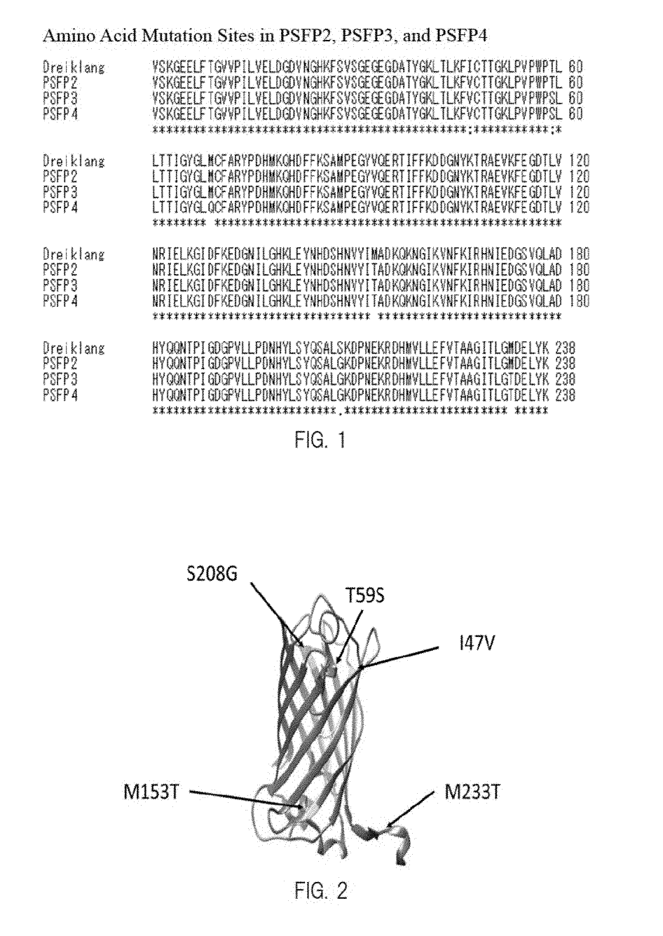

Further two mutations, Mutations 2 and 5 described below, were introduced into PSFP2. Thus, a photo-switching fluorescent protein PSFP3 was produced (a protein consisting of an amino acid sequence of SEQ ID NO: 3). The five mutation sites of Mutations 1 to 5 are shown in FIGS. 1 and 2.

Further a M69Q mutation was introduced into PSFP3 and thereby a photo-switching fluorescent protein PSFP4 was produced (a protein consisting of an amino acid sequence of SEQ ID NO: 4).

### Mutation 1: I47V

### Mutation 2: T59S

### Mutation 3: M153T

### Mutation 4: S208G

### Mutation 5: M233T

### [Production of Bacterial Expression Vector]

Genes (SEQ ID NOS: 5, 6, and 7) that encode PSFPs (PSFP2, PSFP3, and PSFP4, respectively) each were introduced into a bacterial expression vector pRSET_{B} and thereby bacterial expression vectors of PSFPs were produced. Similarly, with respect to Dreiklang, a bacterial expression vector was produced in the same manner.

### [Production of Mammalian Expression Vector]

Genes that encode PSFPs (PSFP2, PSFP3, and PSFP4) each were introduced into a mammalian expression vector pcDNA3 and thereby mammalian expression vectors of PSFPs were produced.

Furthermore, mammalian expression vectors of fusion fluorescent proteins in which the following signal sequence or signal protein was fused with each of PSFPs (PSFP2, PSFP3, and PSFP4) were also produced. That is, 1) a redundant mitochondrial targeting signal derived from the precursor of subunit VIII (COX-VIII) of human cytochrome c oxidase, 2) a Golgi body localization signal sequence of β-N-acetylglucosaminyl-glycopeptide β-1,4-galactosyltransferase, 3) a DNA binding protein H2B, and 4) a nucleolar protein, fibrillarin, were fused with the PSFPs to target mitochondria, Golgi body, nucleus, and nucleolus, respectively. Moreover, mammalian expression vectors of fusion proteins in which β-actin, vimentin, paxillin, zyxin, and clathrin were fused with the PSFPs, respectively, through a 17-amino-acid-long linker sequence (GGSGGSGGSGGSGGQFQ: SEQ ID NO: 8) were also produced.

### [Purification of PSFPs]

PSFPs (PSFP 2, PSFP 3, and PSFP 4) having a polyhistidine tag at the N-terminus each were introduced into the bacterial expression vector pRSET_{B}, which then were expressed in Escherichia coli After culturing them in LB medium at 23° C. for 65 hours, the bacterial cells were disrupted with a French press and the supernatant was purified by a Ni-NTA agarose affinity column (manufactured by Qiagen) and gel filtration with a PD-10 column (manufactured by GE Healthcare) and further purified again using AKTA 10S (GE Healthcare) with a Hiload 200 Superdex 200 pg column.

### [Characterization of PSFPs]

Fluorescence excitation as well as photo-switching on and off of the fluorescence of PSFPs (PSFP2, PSFP3, and PSFP4) were carried out using LED light sources with wavelengths of 475±28 nm, 360±20 nm, and 410±10 nm, respectively. Absorption spectra were measured using a V-630 BIO spectrophotometer (manufactured by JASCO). The fluorescence excitation and fluorescence emission spectra were measured using an F-7000 fluorescence spectrophotometer (manufactured by Hitachi). Molar extinction coefficients each were calculated using the absorbance of a purified protein with a known concentration measured by the Bradford assay. Fluorescence quantum yields were measured using Quantaurus QY-C 11347 (manufactured by Hamamatsu Photonics). In this measurement, the protein absorbance was adjusted to less than 0.05. All the above-mentioned measurements were carried out under physiological pH conditions using proteins contained in 20 mM HEPES buffer. Quantum yields of photo-induced on-off switching were calculated by measuring the irradiation-dependent changes in absorbance with a V-630 BIO spectrophotometer (manufactured by JASCO). Specifically, the method described in Gayda, S., Nienhaus, K & Nienhaus, G. U. Biophysical journal 103, 2521-31 (2012) was followed. For the measurement of the thermal relaxation time of fluorescence from off to on, fluorescence spectra were measured after fluorescence of 10 ml of 20 μM protein solution (20 mM HEPES buffer, pH 7.4) was switched off by light with a wavelength of 438±20 nm before the measurement.

[Rate of Recovery from Off State Due to Thermal Equilibrium]

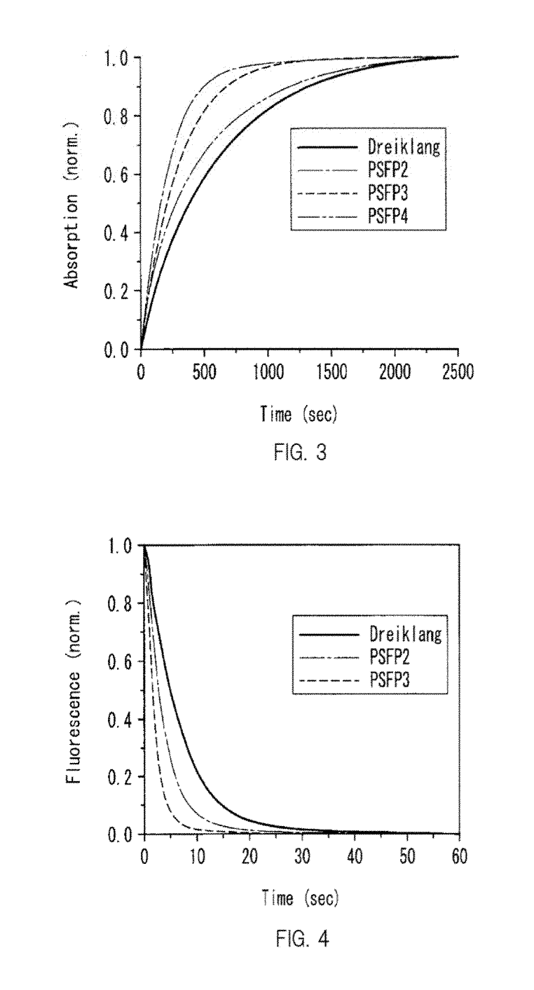

The rate of recovery of fluorescence from the off state to the on state due to thermal equilibrium was measured with respect to PSFP2, PSFP3, and PSFP4, and the rates were compared with that of their counterpart, Dreiklang. Specifically, with respect to the recovery of fluorescence, the absorption spectrum at 511 nm was measured. As a result, the time constants of the rate of recovery due to thermal equilibrium were 218 seconds for PSFP 2, 298 seconds for PSFP 3, 526 seconds for PSFP 4, and 603 seconds for Dreiklang (Table 1, FIG. 3).

TABLE 1
Time Constant of Rate of Recovery
due to Thermal Equilibrium

| Dreiklang | PSFP2 | PSFP3 | PSFP4 |
|---|---|---|---|
| (sec) | (sec) | (sec) | (sec) |
| 603 | 218 | 298 | 526 |

### [Photo-Switching Speed]

The photo-switching characteristics of PSFPs (PSFP 2 and PSFP 3) were compared with those of their counterpart, Dreiklang. The half life (t_{1/2}) of photo-switching fluorescence from on to off was 4.3 seconds for PSFP 2, 2.4 seconds for PSFP 3, and 8.8 seconds for Dreiklang (Table 2, FIG. 4).

TABLE 2

| | Half-life of photo-switching (t½) | | |
|---|---|---|---|
| | Dreiklang | PSFP2 | PSFP3 |
| | (sec) | (sec) | (sec) |
| Switch-Off | 8.8 | 4.3 | 2.4 |

### [Photostability]

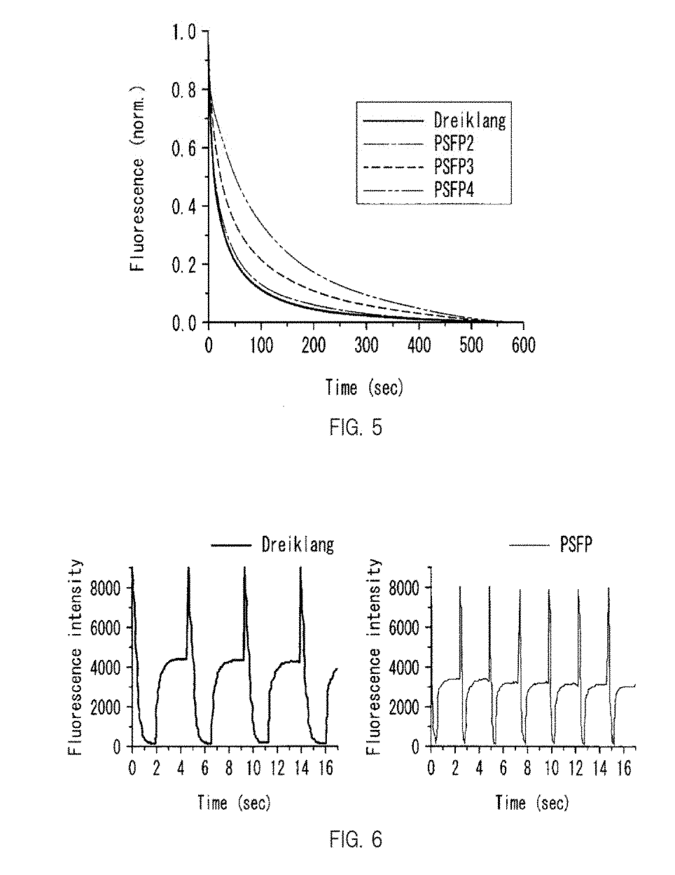

Fluorescence decay due to fluorescence bleaching of PSFPs (PSFP 2, PSFP 3, and PSFP 4) was compared with that of their counterpart, Dreiklang. Specifically, each sample was irradiated with an excitation light and the change in fluorescence intensity was measured. As a result, the half-life (t_{1/2}) of the fluorescence decay was 48 seconds for PSFP 2, 78 seconds for PSFP 3, 123 seconds for PSFP 4, and 43 seconds for Dreiklang able 3, FIG. 5).

TABLE 3
Fluorescence Decay Half-Life (t_{1/2})

| Dreiklang | PSFP2 | PSFP3 | PSFP4 |
|---|---|---|---|
| (sec) | (sec) | (sec) | (sec) |
| 43 | 48 | 78 | 123 |

### [Number of Photo-Switching Cycles]

The improvement in fluorescence on-off speed and the improvement in photostability in the fluorescent protein of Example 1 increased the number of photo-switching (FIG. 6). That is, the number of cycles required until the fluorescence intensity decreased by 50% was 230 cycles for PSFP 3 and 195 cycles for Dreiklang.

### [Localization of Fusion Fluorescent Protein]

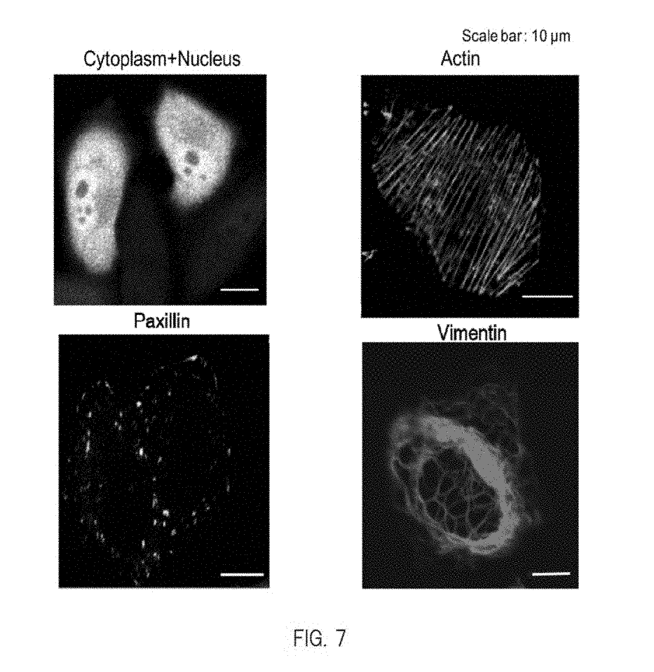

PSFP3 alone or fusion proteins of PSFP3 with β-actin, paxillin, or vimentin were expressed in HeLa cells and observed under a fluorescence microscope. The results are shown in FIG. 7. As shown in FIG. 7, PSFP3 alone was localized in the cytoplasm and nucleus, and the fusion proteins with β-actin, paxillin, or vimentin were localized in actin, paxillin, or vimentin, respectively; and they al exhibited fluorescence.

### [Super-Resolution Imaging by DSSM]

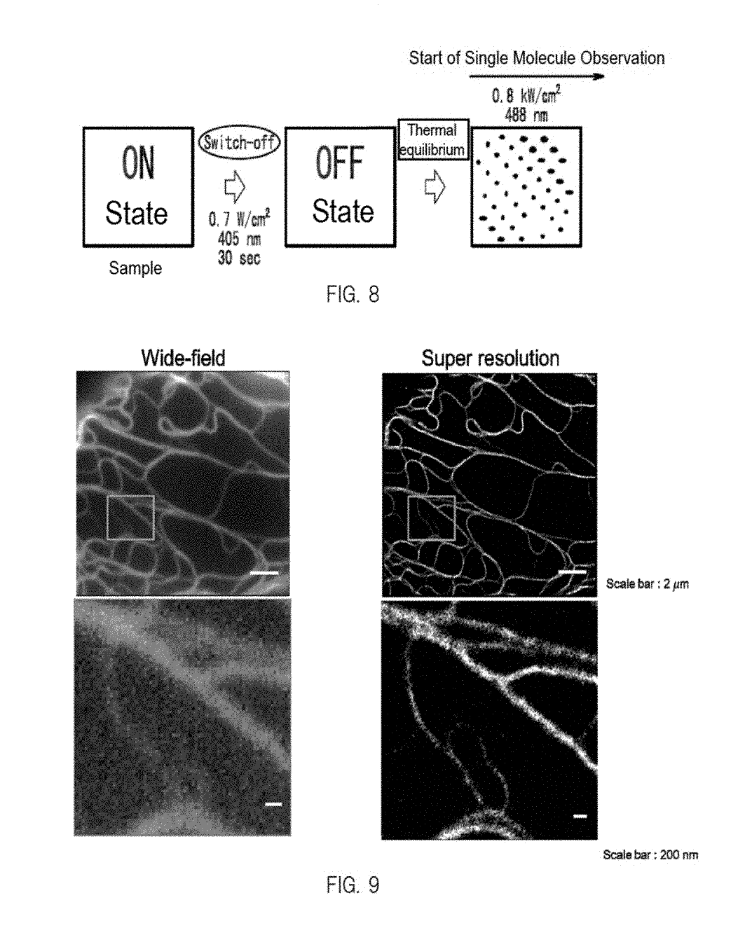

A fusion protein of PSFP3 and vimentin was expressed in HeLa cells and super-resolution imaging was performed by DSSM (decoupled stochastic switching microscopy). The outline of the DSSM imaging is shown in FIG. 8. That is, first, PSFP3 whose fluorescence was on was irradiated with light of 405 nm and thereby the fluorescence was switched off. PSFP3 spontaneously switches fluorescence on due to thermal equilibrium. Next, by irradiation with an excitation light of 488 nm, single molecule fluorescence observation was carried out and at the same time, the fluorescent molecule was bleached, and thereby a single molecule bright spot on the screen was sparsely measured. The result is shown in FIG. 9 together with the wide field of view image (left). In the super-resolution imaging by DSSM of this example, the images were obtained successfully with a spatial resolution of 34 nm and a temporal resolution of 20 seconds, which are higher than the previously reported values of spatial resolution and temporal resolution.

### [Example of Fluorescence Photo-Switching of Localized Fusion Fluorescent Protein]

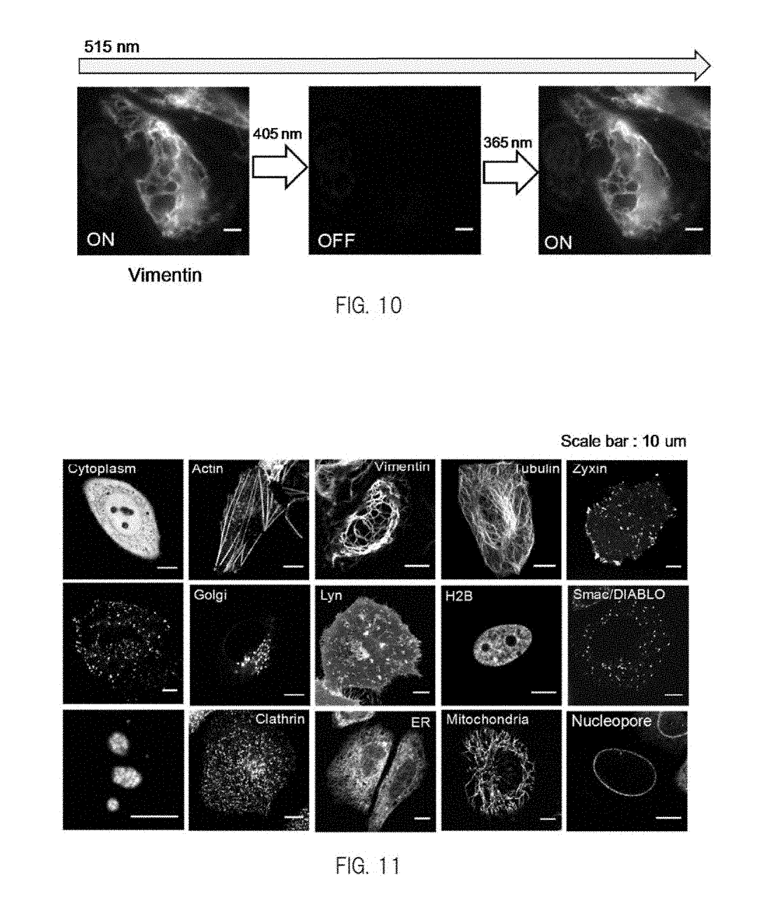

Living HeLa cells in which the vimentin-PSFP3 fusion protein produced above was expressed were subjected to fluorescence photo-switching, which was observed. Specifically, photo-switching was carried out in which while an excitation light of 515 nm was irradiated, fluorescence was switched off with light of 405 nm, and then fluorescence was switched on with light of 365 nm. The result is shown in FIG. 10. As shown in FIG. 10, photo-switching was observed in the fusion fluorescent protein localized in the living HeLa cells.

### [Localization of Fusion Fluorescent Protein (Addition)]

PSFP3 fusion proteins with a localization signal or one of the proteins including tubulin, zyxin, Src tyrosine kinase Lyn, histone H2B, Smac/DIABLO, fibrillarin, clathrin, Golgi body, endoplasmic reticulum (ER), mitochondria, and nuclear pore (Nucleopore) in addition to 6-actin, paxillin, and vimentin were produced, expressed in HeLa cells, and then observed under a fluorescence microscope. The results are shown in FIG. 11. As shown in FIG. 11, each fusion protein was localized and exhibited fluorescence.

### [Adeno-Associated Virus (AAV) Vector That Expresses PSFP]

pAAV-CAG-PSFP3 and pAAV2-hSyn-PSFP3 each having a PSFP3 gene bound to one of two types of promoters (CAG and hSyn) were used to produce AAV vectors. These AAV vectors were infected with primary cultured cells of HEK293T cells and hippocampal neurons, respectively, and thereby expression and fluorescence of PSFP3 were confirmed.

### [LifeAct-PSFP3 Fusion Protein]

An expression vector having a gene that encodes a PSFP3 fusion protein with the peptide LifeAct (J. Riedl et al, Nature Methods 5, 605-607, 2008) that binds to F-actin (a filamentous polymer of G-actin) was produced (LifeAct-PSFP3-pcDNA3).

### [Time-Lapse Super-Resolution Imaging]

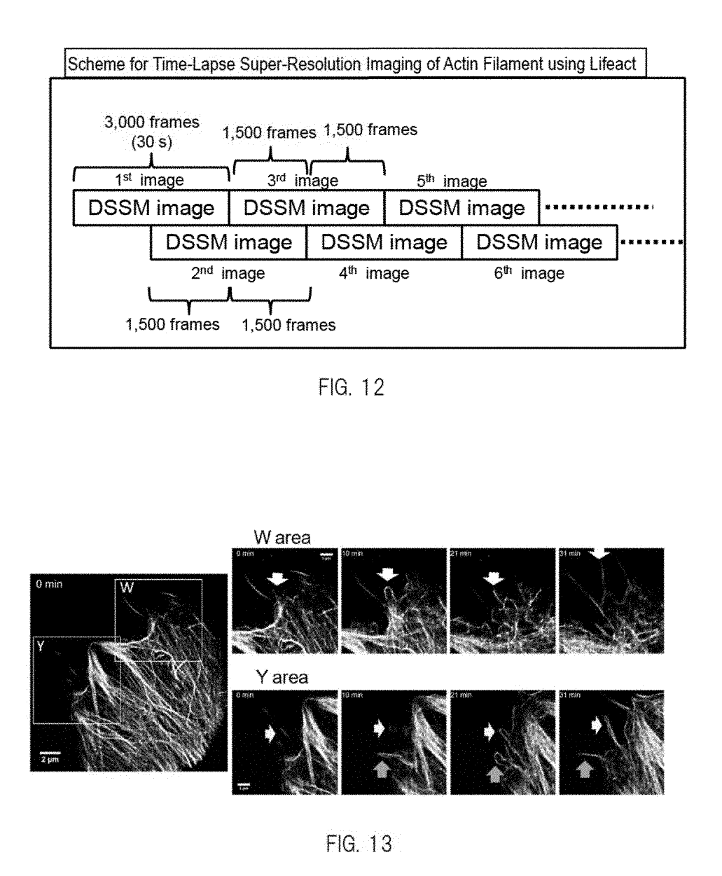

The above-mentioned LifeAct-PSFP3 fusion protein was expressed in living HeLa cells, and time-lapse super-resolution imaging in which DSSM imaging was repeated was performed as shown in FIG. 12. The results are shown in FIG. 13.

As shown in FIG. 13, the morphological change of the actin network were observed by using the time-lapse super-resolution imaging.

### [Sequence Listing Free Text]

SEQ ID NO: 1: Amino acid sequence of Dreiklang

SEQ ID NO: 2: Amino acid sequence of PSFP2

SEQ ID NO: 3: Amino acid sequence of PSFP3

SEQ ID NO: 4: Amino acid sequence of PSFP4

SEQ ID NO: 5: Base sequence of PSFP2

SEQ ID NO: 6 Base sequence of PSFP3

SEQ ID NO: 7: Base sequence of PSFP4

SEQ ID NO: 8: Amino acid sequence of linker

1. A fluorescent protein having an amino acid sequence in which at least an S208G mutation is introduced into an amino acid sequence of SEQ ID NO: 1.

2. The fluorescent protein according to claim 1, wherein the fluorescent protein has an amino acid sequence in which at least three mutations, I47V, M153T, and S208G, are introduced into the amino acid sequence of SEQ ID NO: 1.

3. The fluorescent protein according to claim 1, wherein the fluorescent protein has an amino acid sequence in which at least five mutations, I47V, T59S, M153T, S208G, and M233T, are introduced into the amino acid sequence of SEQ ID NO: 1.

4. The fluorescent protein according to claim 1, wherein the fluorescent protein has an amino acid sequence in which at least six mutations, I47V, T59S, M69Q, M153T, S208G, and M233T, are introduced into the amino acid sequence of SEQ ID NO: 1.

5. The fluorescent protein according to claim 1, wherein the fluorescent protein has an amino acid sequence of any one of SEQ ID NOS: 2 to 4.

6. A fluorescent protein, the fluorescent protein having an amino acid sequence in which one to several amino acids are deleted, substituted, and/or added in an amino acid sequence of the protein according to claim 1, and in the fluorescent protein, a wavelength for switching a non-fluorescent state to a fluorescent state, a wavelength for switching a fluorescent state to a non-fluorescent state, and a wavelength for fluorescence excitation all differing from one another and the rate of recovery from a non-fluorescent state to a fluorescent state due to thermal equilibrium being higher than that of a protein consisting of an amino acid sequence of SEQ ID NO: 1.

7. A fusion protein in which the fluorescent protein according to claim 1 is fused, in the fluorescent protein, a wavelength for switching a non-fluorescent state to a fluorescent state, a wavelength for switching a fluorescent state to a non-fluorescent state, and a wavelength for fluorescence excitation all differing from one another and the rate of recovery from a non-fluorescent state to a fluorescent state due to thermal equilibrium being higher than that of a protein consisting of an amino acid sequence of SEQ ID NO: 1.

8. A DNA comprising a base sequence that encodes the protein according to claim 1.

9. A vector being capable of expressing the protein according to claim 1.

10. A transformant that expresses the protein according to claim 1.

11. An imaging method using the protein according to claim 1.

12. The imaging method according to claim 11, wherein the imaging method is super-resolution fluorescence microscopy imaging.

13. A photochromic material comprising a protein according to claim 1.
